(19) United States
(12) Patent Application Publication
(10) Pub. No.: US 2011/0275085 A1
(43) Pub. Date: Nov. 10, 2011

(54) METHOD FOR DETECTION OF AUTOIMMUNE DISEASES

(75) Inventors: Harri Salo, Helsinki (FI); Jarno Honkanen, Tampere (FI); Outi Vaarala, Helsinki (FI)

(73) Assignee: TERVEYDEN JA HYVINVOINNIN LAITOS, Helsinki (FI)

(21) Appl. No.: 13/132,048

(22) PCT Filed: Dec. 1, 2009
(86) PCT No.: PCT/FI2009/050966

§ 371 Date: Jul. 21, 2011

(30) Foreign Application Priority Data
Dec. 1, 2008 (FI) 20086145

(51) Int. Cl. C12Q 1/68; G06F 19/24

(52) U.S. Cl. 435/6.12; 435/6.17; 435/6.1; 435/6.18; 706/20

(57) ABSTRACT

The present invention relates to the field of diagnostics, especially to the detection of autoimmune diseases such as rheumatoid arthritis. Particularly, the invention provides a method for detecting the presence or absence of rheumatoid arthritis, or of a predisposition therefore or for monitoring rheumatoid arthritis in a subject using expression data of target genes related to immune system and tools of bioinformatics.

### FIELD OF THE INVENTION

The present invention relates to the field of diagnostics, especially to the detection of autoimmune diseases such as rheumatoid arthritis. Particularly, the invention provides a method for detecting the presence or absence of rheumatoid arthritis, or of a predisposition therefor or for monitoring rheumatoid arthritis in a subject using expression data of target genes related to immune system.

### BACKGROUND OF THE INVENTION

Many genes potentially associated with autoimmune diseases are known, and recently it has been suggested that expression profiles of these target genes may be used for assessing the presence of various autoimmune diseases or of a predisposition therefor in a patient (see, e.g., WO 2004/056866, and US 2005/0048574).

Rheumatoid arthritis is an autoimmune disease affecting multiple organs and tissues but is primarily characterised by inflammation in synovial joints causing painful symptoms and leading often to severe disability. Approximately 1% of the population suffers from the disease, and it is about three times more common in women than men. Early and prompt diagnosis of rheumatoid arthritis would be highly beneficial for patients, since best results are achieved if the treatment is initiated at the early stage of the disease. Further, the most effective treatments are aggressive and expensive and thus patients should be correctly diagnosed and treated only when needed.

Rheumatoid arthritis can be difficult to diagnose in its early stages for several reasons. First, there is no single test for the disease. The patient's description of pain, stiffness, and joint function and how these change over time is critical to the physician's initial assessment of the disease. Physical examination of patient, x-rays and laboratory tests such as rheumatoid factor, white blood cell count, erythrocyte sedimentation rate and c-reactive protein provide information of possible arthritis. However, no biomarker has yet been shown to outperform or enhance the predictive accuracy of above mentioned clinical variables that are currently in practice.

Tools of bioinformatics for explaining complex system biology have been used successfully in search of diagnostic measures. Linear regression analysis, artificial neural network (ANNs) and non-linear pattern recognition techniques are rapidly gaining in popularity in medical decision-making. ANNs have been used successfully in, for example, making prediction about the outcome of terminal liver disease (Cucchetti, Vivarelli et al. 2007), in diagnosis of acute myocardial infarction (Heden, Ohlin et al. 1997) and colonic tumors (Selaru, Xu et al. 2002) as well as in analyses (Papadopoulos, Fotiadis et al. 2005) and treatment (Eden, Ritz et al. 2004) of breast cancer. ANNs have also been used in prediction of acute pancreatitis and pancreatic cancer (reviewed in (Bartosch-Harlid, Andersson et al. 2008). The aim of the present study was to search for a method to clinically distinguish rheumatoid arthritis (RA) from non-RA patient. The method utilise quantitative RT-PCR data of immune related genes from the whole blood sample. The analysis of this data with an ensemble of prediction methods, for example, ANN, linear regression, linear discriminant, k-nearest neighbor (KNN), and decision tree is advantageous, since these differently working tools can provide more robust prediction results to identify RA and non-RA.

US 2005/0003394 discloses that it is possible to detect rheumatoid arthritis related gene transcripts from blood samples. Groups of genes associated with rheumatoid arthritis or corresponding microarrays are disclosed, e.g., in US 2008/0108077, US 2006/0127963, US 2005/0048574, US 2007/0196835, US 2008/0113346, US 2003/0154032, US 2007/0298518, and WO 2007/137405. However, there is still a continuing need for novel methods enabling rapid and accurate diagnosis of patients with rheumatoid arthritis. The present invention provides a pattern of clinical markers related to immune system and tools of bioinformatics for efficient assessment of rheumatoid arthritis from a whole blood sample obtained from a patient suspected to have rheumatoid arthritis or to be prone to develop the disease.

### DETAILED DESCRIPTION OF THE INVENTION

The present invention is directed to the detection of the presence or absence of an autoimmune disease in a subject. Autoimmune diseases to which the present invention is related are rheumatoid diseases such as rheumatoid arthritis and ankylosing spondylitis, and inflammatory bowel diseases. In particular, the present invention provides a method for detecting the presence or absence of rheumatoid arthritis. In another embodiment the method can be used for assessing a predisposition for rheumatoid arthritis and thus it would be possible to detect those subjects who are prone to develop rheumatoid arthritis. In another embodiment, the method can also be used for monitoring the progress of rheumatoid arthritis in a patient thus, e.g., enabling a physician to follow the effect of prescribed medication.

In detail, the method of the invention comprises the steps of:

a) isolating total RNA or mRNA from a whole blood sample obtained from a patient;
b) quantifying from the total RNA or mRNA obtained from step a) the amount of mRNA products of the genes selected at least partly from the group consisting of: C3, CR1, CD25, Foxp3, Galectin-9, GATA-3, GITR, ICOS, IFN-gamma, IL-2, IL-4R, IL-12Rb12, INOS, TBET and TIM-3; and
c) inputting the data obtained from step b) to a classifier to detect the presence or absence of the autoimmune disease of interest in the subject or if the subject is prone to suffer from said autoimmune disease, wherein said classifier is trained with data from plurality of subjects with a known status i.e. healthy controls and patients suffering from said autoimmune disease, and the training data is based on mRNA expression results of essentially same genes selected in step b).

Preferably, the amount of mRNA products in step b) is quantified at least from the genes selected from any of the groups consisting of:
a) IFN-gamma and CR1;
b) IFN-gamma, CR1, and GITR;
c) IFN-gamma, CR1, and C3;
d) IFN-gamma, CR1, GITR, and C3;
e) CR1 and GITR; and
f) CR1, GITR and C3.

Further groups for step b) consist of:
g) IFN-gamma, CR1 and TIM-3;
h) IFN-gamma, C3 and TIM-3; and
i) IFN-gamma, CR1, C3, and TIM-3,
   which are preferably analysed by ANN in step c).

Still one further group consists of:
j) IFN-gamma, Foxp3, and GITR,
   and is preferably analysed by linear regression or linear discriminant in step c).

Majority of the marker genes in the present study are T cell markers (see Table 1). Evidence exists that CD4 T cells likely play a dominant role in the immunopathogenesis of autoimmune inflammatory rheumatic disease, such as rheumatoid arthritis (for review see (Skapenko, Lipsky et al. 2006). CD4 T cells that emerge from thymus belong to the naive T cell pool. Upon proper activation, naive T cells proliferate and differentiate into specific effector cells. CD4 T cells can differentiate into specialized effector cells classified as Th1, Th2, Th17, or Treg cells. For each CD4 T cell differentiation programme, specific transcription factors have been identified as master regulators. TBET is transcription factor for Th1, GATA-3 for Th2, ROR-gamma t for Th17 and Foxp3 for Treg cells. In the present study all these transcription factors were studied except ROR-gamma t that was too low in copy number to be reliably detectable from the majority of samples.

In addition, two genes of complement cascade, namely complement component 3 (C3) and complement receptor 1 (CR1), were included in the present study. There is convincing evidence that both classical and alternative complement pathways are pathologically activated during RA (Okroj, Heinegard et al. 2007). Central to complement activation is the cleavage of C3. Complement cascade is rapidly activated and potentially destructive also to host. Thus proper regulation of complement activation is essentially important in the inflammation. CR1 is a membrane-bound complement inhibitor belonging to regulators of complement activation (RCA) gene cluster.

In another embodiment of the invention, step b) is preferably performed by RT-PCR, such as reverse transcription real-time quantitative polymerase chain reaction (RTqPCR).

However, an important challenge of quantitative gene expression studies based on RT-PCR is to extract sufficient usable messenger ribonucleic acid (mRNA), to avoid degradation and permit analysis for calculation of exact numbers of transcript. The processes of sample collection, transport, processing and storage may result in significant degradation of mRNA (Hartel, Bein et al. 2001). Because of the lability of mRNA in clinical samples, it is essential that the integrity of the mRNA is assessed before proceeding with downstream applications such as reverse transcription real-time quantitative polymerase chain reaction (RTqPCR) and micro-array analyses. Both techniques are highly sensitive and rely on meticulous and consistent sample processing (Lockhart and Winzeler 2000; Stordeur, Zhou et al. 2003). The correct interpretation of transcript abundance requires stabilisation of the transcriptome at the point of sample collection, through storage and transport, in order for gene expression to be detected in a reproducible manner (Thach, Lin et al. 2003).

Good quality RNA for the present method may preferably be obtained by using a kit of the PAXgene™ Blood RNA System (PreAnalytiX, QIAGEN, Germany) including a stabilizing additive in an evacuated blood collection tube called the PAXgene™ Blood RNA Tube, and also sample processing reagents in the PAXgene™ Blood RNA Kit. The additive in the PAXgene™ tube reduces RNA degradation of 2.5 mL of blood in the evacuated tube, and furthermore, the RNA in whole blood has been shown to be stable at room temperature for 5 days, following storage for up to 12 months at −20° C. and −80° C., and also after repeated freeze-thaw cycles (Rainen, Oelmueller et al. 2002).

The quantities of the specific gene expression can be analyzed by a comparative threshold cycle (Ct) method of relative quantification, and for this method gene expression results should be normalized. In normalization, the CT value of a known housekeeping gene, such as 18S (Hs99999901_s1), ACTB (Hs99999903_m1), B2M (Hs99999907_m1), GAPDH (Hs99999905_m1), GUSB (Hs99999908_m1), HMBS (Hs00609297_m1), HPRT1 (Hs99999909_m1), IPO8 (Hs00183533_m1), PGK1 (Hs99999906_m1), POLR2A (Hs00172187_m1), PPIA (Hs99999904_m1), RPLP0 (Hs99999902_m1), TBP (Hs99999910_m1), TFRC (Hs99999911_m1), UBC (Hs00824723_m1), YWHAZ (Hs00237047_m1), or any other gene or their combination is subtracted from the marker gene CT values resulting in delta CT (dCT) value. These Delta CT values are then used in statistical analyses. However, it is also possible to use plain CT values, i.e. normalization to zero, as starting material for statistical analyses.

In the present invention, step c) of the method is performed by computational analysis of the results. Said computational analysis is preferably performed by linear prediction methods, including but not restricted to regression analysis, linear discriminant analysis or nonlinear prediction methods, including but not restricted to an artificial neural network (ANN). These and other statistical analysis methods useful in the present invention are described, e.g., in the following patent applications: WO 01/31579; WO 02/06829, WO 02/42733, US 2004/0073376, US 2004/0137471, US 2006/0195269, US 2007/0198198 and US 2007/0094168.

In the preferred embodiment of the invention, the statistical analysis method is divided into the learning phase and the classification phase. In the learning phase, a learning algorithm is applied to a data set that includes members of the different classes that are meant to be classified, for example, data from a plurality of samples taken from patients with diagnosed rheumatoid arthritis and data from a plurality of samples taken from healthy controls, i.e. persons who do not suffer from an autoimmune disease or other ongoing inflammatory disease. The methods used to analyze the data include, but are not limited to, artificial neural network, regression, Fisher's discriminant, and classification and regression tree analysis. These methods are described, for example, in the prior art publications listed above. The learning algorithm produces a classifying algorithm. The classifier is keyed to elements of the data, such as particular markers and particular intensities of markers, usually in combination, that can classify an unknown sample into one of the two classes. The classifier is then used for diagnostic testing. Both commercial software and freeware is readily available to analyze such patterns in data.

The method of the invention thus uses a classifier for detecting the presence or absence of an autoimmune disease in a subject. The classifier can be based on any appropriate pattern recognition method (i.e. a statistical method) that after receiving input data comprising a gene marker profile based on mRNA expression results is able to provide output data indicating the presence or absence of an autoimmune disease in a subject. The classifier is first trained with training data based on mRNA expression results from plurality of subjects with a known status, i.e. healthy controls and patients suffering from an autoimmune disease of interest. The training data comprise for each subject: a) a marker profile comprising measurements of gene products in an appropriate biological sample, e.g., a whole blood sample taken from the subject; and b) information regarding the status of the subject, i.e. the subject is suffering from the autoimmune disease of interest or he/she is a healthy control. A trained classifier can then be used for generating an indication of the presence or absence of an autoimmune disease in any further subject, when the input data given to the classifier is derived from an appropriate sample taken from said further subject and comprises mRNA expression results of marker genes used also in the training phase.

In the specific embodiment of the invention, the following approach was employed to identify gene transcripts whose changes in expression levels were most highly correlated with rheumatoid arthritis. To initially build and train the classifiers, the expression patterns of the controls and the expression patterns from patient samples were used as the training set. Then MLP-ANN with maximum 6 hidden nodes, linear discriminant, linear regression, KNN and decision tree were used to identify genes with expression levels most highly correlated with the classification vector characteristic of the training set. Predictor sets containing all possible gene combinations were then evaluated by “leave one out cross validation” (LOOCV) to identify the predictor set with the highest accuracy for classification of the samples in the training set. IFN-gamma, CR1, GITR, and C3 were the top genes that were present in the highest accuracy classifiers more often than other genes. Further, IFN-gamma, Foxp3, and GITR were the top genes in linear discriminant and linear regression methods as well as IFN-gamma, CR1, C3, and TIM-3 in MLP-ANN.

In this invention, good results for data analysis were obtained with linear regression and linear discriminant methods followed by ANN as measured with leave-one-out-cross-validation (LOOCV) and receiver order characteristics (ROC) analysis. Correlation of the expression results with rheumatoid arthritis is established, when the ROC analysis yields an area under the curve of at least 0.8, preferably at least 0.9 and more preferably at least 0.91 or 0.92.

Particularly, a preferred embodiment of the invention is a method wherein the amount of mRNA products of the genes comprising at least the group consisting of: C3, CR1, Foxp3, GITR, ICOS, IFN-gamma, IL-2, IL-12Rb12, and TIM-3, is detected, and the data obtained is inputted to a classifier, which is based on a linear prediction method, such as a linear regression model including regression analysis and linear discriminant analysis.

All publications and patent applications cited in this specification are herein incorporated by reference as if each individual publication or patent application were specifically and individually indicated to be incorporated by reference. The following Experimental Section will assist those skilled in the art to better understand the invention and its principles and advantages. It is intended that the Experimental Section be illustrative of the invention and not limit the scope thereof.

### EXPERIMENTAL SECTION

### Materials and Methods

Peripheral whole blood sample (2.5 mL) was taken from newly diagnosed rheumatoid arthritis patients (n=36) and healthy adults (n=38) into the PAXgene Blood RNA Tubes (Becton Dickinson). The samples were gently inverted and let to stay at room temperature for two hours, then stored at −20° C. for maximum 6 months.

Prior to RNA extraction the samples were removed from −20° C. and incubated at room temperature for 2 hours to ensure complete lysis. Total RNA was purified using the PAXgene Blood RNA System Kit (Qiagen) according to the manufacturer's instructions including an added DNAse option (Qiagen). Yield and purity of RNA were determined using a NanoDrop ND-1000 Spectrophotometer (Labtech International, Ringmer, UK).

Reverse transcription of RNA at concentration of 10 ng/μl was carried out using a TaqMan Reverse Transcription reagents (Applied Biosystems, Foster City, Calif., USA).

Real-time quantitative PCR was performed with an ABI 7700 Sequence Detection System (Applied Biosystems), using the TaqMan Universal PCR Master Mix protocol. Primers and TaqMan probe for the human genes were obtained from Applied Biosystems as a TaqMan Gene Expression Assay (Table 2). The 52 μl reaction mix was pipetted in PCR plate in 15 μl triplicates. Reaction mix consisted of 2 μl of the cDNA product except 20 μl for INOS and IL-2, 26 μl of TaqMan 2× Universal PCR Mastermix and 2.6 μl of the 20× TaqMan Gene Expression Assay mastermix and rest of the reaction volume was deionised water. The PCR cycling parameters were set as follows: 95° C. for 10 minutes followed by 40 cycles of 95° C. for 15 seconds and 60° C. for one minute. An exogenous cDNA pool calibrator was collected from PHA stimulated PBMC and considered as an interassay standard, that was run in each plate.

The quantities of the specific gene expression were analyzed by a comparative threshold cycle (Ct) method of relative quantification. In normalization the CT value of the sample housekeeping gene18S was subtracted from the target gene CT values resulting delta CT (dCT) value. Delta CT values were used in statistical analyses.

In INOS total 17 of 72 samples were beyond reliable detection limit. Detection was considered as reliable, if all triplicate runs gave CT value and their SD<1. Samples beyond detection limit were given artificial dCT value (26.5), that in the present study stands for the lowest gene copy level for INOS.

### Data Analysis

The data set consisted on 15 genes and housekeeping gene 18S measured from 74 samples (36 cases and 38 controls).

The aim of the analysis was to find the best classifier for separate cases or controls. We employed leave-one-out-cross-validation schema for a spectrum of prediction methods (neural networks, decision trees, k-nearest neighbourhood, linear discriminant and linear regression) that have been individually used in various diagnostic studies.

### ANN

It is known that ANNs are sensitive to the input variable combinations and cannot perform automatic dimension reduction (Haykin 1998) that, for example, decision trees are able to do. Therefore, we employed a strategy where we used all 32767 gene combinations to train the ANNs. The ANN method we used was the multi-layer perceptron (MLP) neural network (Haykin, 1998). The crucial parameter in MLPs is the number of hidden nodes. For each gene combination, we tested the number of hidden nodes equaling the number of input genes except if the number of input genes was more than 6, only 6 hidden nodes were tested. Thus, we trained altogether 193952 MLP neural networks. For each network, the input data (LOOCV training data) 95% of the data were used in training the MLP network and 5% to test when to stop MLP training in order to avoid overfitting. After training an MLP network it was applied to the left-out sample. The other parameters for the MLP networks were as follows. We used tansig transformation function, and the output was rounded to the closest outcome (−1 denoting controls and +1 denoting cases). For the neural networks, the training data were scaled between −1 and 1 (Haykin 1998) inside the LOOCV loop, and the transformation parameters were stored. The LOOCV sample was scaled using the stored scaling parameters and then applied to the MLP neural network. All possible gene combinations were analyzed with the LOOCV using the MLP network with the above mentioned parameters. The MLP classifiers were constructed in MATLAB v.7.4.0.287 and neural networks toolbox v.5.0.2 using the same seed in the initialization of the network (9.85337161E8). The network was created with ‘newff’ command and the fraction of the data points used in the test set was 5%. The test set was used to monitor possible over-learning and stop training if such phenomenon was detected. The initiated network was trained with the command ‘train’. Class for the left-out sample was determined with the trained network and the command ‘sim’.

The parameters for the other classifiers were as follows:
1. Discriminant analysis: MATLAB command ‘classify’ was used.
2. Regression analysis: MATLAB command ‘regress’ (the data matrix was added with a column full of ones to account for the constant term in the regression equation.)
3. kNN: We built the kNN classifier with ‘correlation’ distance measure and ‘volumetric’ final decision method.
4. Decision tree: We used classification tree algorithm with MATLAB function ‘treefit’ with Gini index splitting criterion and at least 15 observation was needed for splitting.

We used ROC analysis for the LOOCV estimates to identify the best classifier. The criterion was the area under curve (AUC). The AUC is between 0 and 1, where 1 represents perfect test and 0.5 worthless test. Another criterion was accuracy, i.e., number of correctly classified samples as shown in Table 3.

Clinically reasonable classifiers were obtained both with linear discriminant and linear regression methods as well as with artificial neural network (ANN) method as measured with leave-one-out-cross-validation (LOOCV) and receiver order characteristics (ROC) analysis (Table 3).

Linear regression forms a relationship between independent variables (X, genes) dependent variable (Y, presence or absence of RA) using linear regression equation (Hastie, Tibshirani et al. 2001). Mathematically, y=Xb, where X is an n-by-p design matrix, with rows corresponding to observations and columns to predictor variables, y is an n-by-1 vector of response observations and b regression coefficients typically estimated with least-square analysis method (the first column of X is full of ones to ensure that the model contain a constant term). Here, for the best linear regression (Table 3), the b vector is

| Coefficient | Gene |
|---|---|
| 0.4865 | constant term |
| −0.1321 | Foxp3 |
| −0.2120 | TIM-3 |
| −0.1806 | IFN-gamma |
| 0.1463 | IL-2 |
| 0.1671 | IL-12Rβ2 |
| 0.0921 | GITR |
| 0.0692 | ICOS |
| 0.1521 | C3 |
| −0.2566 | CR1 |

Linear discriminant analysis aims at finding a linear combination of variables that separate the best two output classes (here, RA and healthy). The linear discriminant function is defined as

$$separation = \frac{\vert {\overline{y}}_{1} - {\overline{y}}_{2} \vert}{{s}_{y}} , \\ where {s}_{y}^{2} = \frac{{\sum }_{j = 1}^{{n}_{1}} {( {y}_{1 j} - {y}_{1} )}^{2} + {\sum }_{J = 1}^{{n}_{2}} {( {y}_{2 j} - {y}_{2} )}^{2}}{{n}_{1} + {n}_{2} - 2}$$

is the pooled estimate of the variance. The output of linear discriminant is a covariant matrix. Here the best classifier was obtained with the genes shown at Table 3.

TABLE 1
Marker genes.

| Gene | Gene product | Gene ID |
|---|---|---|
| Foxp3 | forkhead box P3 | NM_014009.2 |
| TBET | T-box 21 | NM_013351.1 |
| GATA-3 | GATA binding protein 3 | NM_001002295.1 |
| TIM-3 | hepatitis A virus cellular | NM_032782.3 |
| | receptor 2 | |
| Galectin-9 | lectin, galactoside-binding, | NM_009587.2 |
| | soluble, 9 (Galectin-9) | |
| IFN-gamma | interferon, gamma | NM_000619.2 |
| CD25 | interleukin 2 receptor, alpha | NM_000417.1 |
| GITR | tumor necrosis factor receptor | NM_148901.1 |
| | superfamily, member 18 | |
| ICOS | inducible T-cell co-stimulator | NM_012092.2 |
| IL-2 | interleukin 2 | NM_000586.3 |
| IL-4R | interleukin 4 receptor | NM_001008699.1 |
| IL-12Rβ2 | interleukin 12 receptor, beta 2 | NM_001559.2 |
| INOS | nitric oxide synthase 2A | NM_000625.3 |
| | (inducible) | |
| C3 | complement component 3 | NM_000064.2 |
| CR1 | complement component | NM_000573.3 |
| | (3b/4b) receptor 1 | |
| 18S | Eukaryotic 18S rRNA | X03205.1 |

It is noted that the sequences of the marker genes listed in Table 1 are available in the public databases. The table provides the accession number and name for each of the sequences. The sequences of the genes in GenBank are herein expressly incorporated by reference in their entirety as of the filing date of this application (see www.ncbi.nlm.nih.gov).

TABLE 2
Assay IDs of TaqMan ® Gene Expression Assays by Applied
Biosystems and related human gene

| Assay ID | gene |
|---|---|
| Hs99999901_s1 | 18S (housekeeping) |
| Hs00163811_m1 | C3 |
| Hs00166229_m1 | CD25 |
| Hs00559348_m1 | CR1 |
| Hs00203958_m1 | Foxp3 |
| Hs00371321_m1 | Galectin-9 |
| Hs00231122_m1 | GATA-3 |
| Hs00188346_m1 | GITR |
| Hs00359999_m1 | ICOS |
| Hs00174143_m1 | IFN-gamma |
| Hs00155486_m1 | IL-12Rβ2 |
| Hs00174114_m1 | IL-2 |
| Hs00166237_m1 | IL-4R |
| Hs00167248_m1 | INOS |
| Hs00203436_m1 | TBET, tbx21 |
| Hs00262170_m1 | TIM-3, havcr2 |

TABLE 3
Best classifiers to separate cases from controls

| | AUC | Mean accuracy | Gene number |
|---|---|---|---|
| Linear regression | 0.915 | 0.946 | 9 (*) |
| Linear discriminant | 0.915 | 0.946 | 9 (**) |
| MLP ANN1 | 0.913 | 0.919 | 11 (***) |
| MLP ANN2 | 0.906 | 0.932 | 10 (****) |

(*) gene set for linear regression was Foxp3, TIM-3, IFN-gamma, IL-2, IL-12Rβ2, GITR, ICOS, C3, and CR1.
(**) gene set for linear discriminant was Foxp3, TIM-3, IFN-gamma, IL-2, IL-12Rβ2, GITR, ICOS, C3, and CR1.
(***) MLP ANN1 used genes GATA-3, Galectin-9, IFN-gamma, CD25, IL-12Rβ2, GITR, ICOS, IL-4R, C3, CR1, and INOS
(***) MLP ANN2 used genes Foxp3, TBET, GATA-3, TIM-3, IFN-gamma, CD25, IL-2, GITR, ICOS, and CR1

### REFERENCES

Bartosch-Harlid, A., B. Andersson, U. Aho, J. Nilsson and R. Andersson (2008). “Artificial neural networks in pancreatic disease.” Br J Surg 95(7): 817-26.
Cucchetti, A., M. Vivarelli, N. D. Heaton, S. Phillips, F. Piscaglia, L. Bolondi, G. La Barba, M. R. Foxton, M. Rela, J. O'Grady and A. D. Pinna (2007). “Artificial neural network is superior to MELD in predicting mortality of patients with end-stage liver disease.” Gut 56(2): 253-8.
Eden, P., C. Ritz, C. Rose, M. Ferno and C. Peterson (2004). ““Good Old” clinical markers have similar power in breast cancer prognosis as microarray gene expression profilers.” Eur J Cancer 40(12): 1837-41.
Hartel, C., G. Bein, M. Muller-Steinhardt and H. Kluter (2001). “Ex vivo induction of cytokine mRNA expression in human blood samples.” J Immunol Methods 249(1-2): 63-71.
Hastie, T., R. Tibshirani and J. Friedman (2001). The elements of statistical learning: data mining, interference, and prediction, Springer.
Haykin, S. (1998). Neural Networks: A Comprehensive Foundation, Prentice Hall.
Heden, B., H. Ohlin, R. Rittner and L. Edenbrandt (1997). “Acute myocardial infarction detected in the 12-lead ECG by artificial neural networks.” Circulation 96(6): 1798-802.
Lockhart, D. J. and E. A. Winzeler (2000). “Genomics, gene expression and DNA arrays.” Nature 405(6788): 827-36.
Okroj, M., D. Heinegard, R. Holmdahl and A. M. Blom (2007). “Rheumatoid arthritis and the complement system.” Ann Med 39(7): 517-30.
Papadopoulos, A., D. I. Fotiadis and A. Likas (2005). “Characterization of clustered microcalcifications in digitized mammograms using neural networks and support vector machines.” Artif Intell Med 34(2): 141-50.
Rainen, L., U. Oelmueller, S. Jurgensen, R. Wyrich, C. Ballas, J. Schram, C. Herdman, D. Bankaitis-Davis, N. Nicholls, D. Trollinger and V. Tryon (2002). “Stabilization of mRNA expression in whole blood samples.” Clin Chem 48(11): 1883-90.
Selaru, F. M., Y. Xu, J. Yin, T. Zou, T. C. Liu, Y. Mori, J. M. Abraham, F. Sato, S. Wang, C. Twigg, A. Olaru, V. Shustova, A. Leytin, P. Hytiroglou, D. Shibata, N. Harpaz and S. J. Meltzer (2002). “Artificial neural networks distinguish among subtypes of neoplastic colorectal lesions.” Gastroenterology 122(3): 606-13.
Skapenko, A., P. E. Lipsky and H. Schulze-Koops (2006). “T cell activation as starter and motor of rheumatic inflammation.” Curr Top Microbiol Immunol 305: 195-211.
Stordeur, P., L. Zhou, B. Byl, F. Brohet, W. Burny, D. de Groote, T. van der Poll and M. Goldman (2003). “Immune monitoring in whole blood using real-time PCR.” J Immunol Methods 276(1-2): 69-77.
Thach, D. C., B. Lin, E. Walter, R. Kruzelock, R. K. Rowley, C. Tibbetts and D. A. Stenger (2003). “Assessment of two methods for handling blood in collection tubes with RNA stabilizing agent for surveillance of gene expression profiles with high density microarrays.” J Immunol Methods 283(1-2): 269-79.

1.-15. (canceled)

16. Method for detecting the presence or absence of rheumatoid arthritis, or of a predisposition therefor in a subject, the method comprising the steps of:
a) isolating total RNA or mRNA from a whole blood sample obtained from a subject;
b) quantifying from the total RNA or mRNA obtained from step a) the amount of mRNA products of the genes comprising at least the group consisting of: C3, CR1, Foxp3, GITR, ICOS, IFN-gamma, IL-2, IL-12Rβ2, and TIM-3; and
c) inputting the data obtained from step b) to a classifier trained to detect the presence or absence of said autoimmune disease in the subject or if the subject is prone to suffer from said autoimmune disease.

17. The method according to claim 16, wherein said classifier has been trained with data from plurality of subjects with a known status, i.e. healthy controls and patients suffering from said autoimmune disease, and the training data is based on mRNA expression results of essentially same genes selected in step b).

18. The method according to claim 16, wherein further target genes for step b) can be selected from the group consisting of: CD25, Galectin-9, GATA-3, IL-4R, INOS and TBET.

19. The method according to claim 16, wherein step b) is performed by reverse transcription real-time quantitative polymerase chain reaction (RTqPCR).

20. The method according to claim 16, wherein said classifier in step c) is a linear prediction method.

21. The method according to claim 20, wherein said linear prediction method is linear regression model including regression analysis and linear discriminant analysis.

22. The method according to claim 16, wherein the method is used for monitoring the progress of rheumatoid arthritis in a patient.

23. Method for constructing a classifier for the detection of the presence or absence of rheumatoid arthritis, or of a predisposition therefor in a subject, the method comprising the steps of:
a) selecting at least the genes C3, CR1, Foxp3, GITR, ICOS, IFN-gamma, IL-2, IL-12Rβ2, and TIM-3;
b) isolating total RNA or mRNA from a whole blood sample obtained from plurality of subjects comprising healthy controls and patients known to suffer from rheumatoid arthritis;
c) quantifying from the total RNA or mRNA obtained from step b) the amount of mRNA products of the genes selected in step a) to provide test data comprising mRNA profiles;
d) inputting the test data to multiple data classifiers;
e) combining the results of step d) to obtain a trained classifier capable to detect the presence or absence of said autoimmune disease based on essentially similar mRNA profile as in step
c) obtained from a further patient sample not used in the training of the classifier.

24. The method according to claim 23, wherein said multiple data classifiers of step d) comprises artificial neural networks, classification and regression trees, k-nearest neighbor classification, and regression.
